(19) United States
(12) Patent Application Publication
(10) Pub. No.: US 2007/0269853 A1
(43) Pub. Date: Nov. 22, 2007

(54) Integrated Device for Diagnostic Analyses, and Relative Method

(75) Inventor: Paolo Galiano, Padova (IT)

Correspondence Address: HAMRE, SCHUMANN, MUELLER & LARSON, P.C., P.O. BOX 2902, MINNEAPOLIS, MN, 55402-0902, US

(73) Assignee: ALIFAX TECHNOLOGY SRL, Nimis(UD) (IT)

(21) Appl. No.: 11/661,042

(22) PCT Filed: Aug. 12, 2005
(86) PCT No.: PCT/EP05/53968

§ 371 Date: Feb. 22, 2007

(30) Foreign Application Priority Data
Aug. 25, 2004 (IT) UD2004A000170

(51) Int. Cl. C12Q 1/06; C12M 3/00

(52) U.S. Cl. 435/39; 435/288.2

(57) ABSTRACT

An integrated device for diagnostic analyses used to verify the presence of bacteria in at least a biological sample mixed with a eugonic culture broth, in order to identify the type of bacteria, and to test a series of antibiotics, identifying those effective to determine the antibiotic therapy. The device comprises first examination means to verify the presence of bacteria so as to define corresponding positive biological samples and identify the type of bacteria present in the positive biological samples to define said group of antibiotics, and second examination means to verify the sensitive or resistant response of each positive biological sample to a series of antibiotics of the group of antibiotics defined by the first examination means.

### FIELD OF THE INVENTION

The present invention concerns an integrated device and the relative method to perform diagnostic analyses on a biological sample. The invention is used to verify the presence in said sample of one or more bacteria and to identify the type thereof, in order to test the appropriate antibiotics to be matched with the bacterium identified in order to establish the possible antibiotic therapy.

The biological sample to be analyzed, or primary biological sample, can be for example urine, cerebrospinal liquid, catarrh, diluted blood or other.

### BACKGROUND OF THE INVENTION

In the field of diagnostic analyses various techniques are known to verify the presence of bacteria in a biological sample, to identify the type of bacteria and to determine a group of antibiotics efficacious in contrasting the bacterial growth of the type identified. This last operation is called technically “sensitivity test to antibiotics”.

Known techniques for doing the sensitivity test to antibiotics provide to verify the functionality of the antibiotics in colonies of isolated bacteria, and therefore presuppose long, previous isolation procedures, to which must also be added the time required for the subsequent verification of the functionality of the antibiotics.

Another disadvantage of known systems is the prevalent use of analysis techniques of a biochemical type.

Especially for serious infections, a long time between the bacterial growth and the sensitivity test to antibiotics can be excessive and entail dangers for the patient. It is common use in medical circles to give the patient, in whom positivity has been found, a wide spectrum antibiotic, that is, one that covers a large number of types of bacteria, in order to reduce the therapy times.

One disadvantage of wide spectrum antibiotics is that, although they are efficacious in contrasting bacterial growth, it may happen that not only not all the bacterial colonies are eliminated, but also the bacteria of the surviving colonies may become resistant to the selected antibiotic and they proliferate, thus increasing the infection.

An example of the above-cited prior art is disclosed in the documents U.S. Pat. No. 5,863,754 and U.S. Pat. No. 6,107,082, which have the same owner, the same inventor and refer substantially to the same machine, and disclose a method and an apparatus for detecting the type of a bacterial colony in a biological sample and the related sensitivity to the antibiotics.

This prior art apparatus is designed to operate on a biological sample which is already known as containing a significant bacterial charge, because the biological samples come from a screening step, executed in another place and with other means, wherein the samples have been screened and selected between positive samples and negative samples. Only the positive samples, i.e. the samples which are supposed to contain the bacterial colony, are manually introduced into a primary receiver, and then processed in order to measure the growth curves of the different kind of bacterial colonies for identifying the bacterial colonies present in the samples.

The preliminary step of screening between positive and negative samples entails very long times, and possibilities of errors during the transfer of the samples from the screening step to the bacterial identification step.

Moreover, this prior art apparatus entails manual operations for inoculating the positive samples in the containers.

Purpose of the invention is to achieve an integrated device for diagnostic analyses of a biological sample able to offer a high level of automation and speed of execution, and able to verify, in a short time, first of all the positivity of the sample, to identify the type of bacteria, at least by typology, for example coccus or bacillus, and subsequently to perform the sensitivity test to antibiotics only to the samples found positive.

In particular, a specific purpose of the present invention is to achieve a method and a device which do not require a preliminary and separate step for screening between positive (i.e. contaminated with bacterial colonies) and negative samples, but which are able to operate on any kind of sample of which, first of all the presence, and then the kind, of bacterial colonies are to be identified.

The Applicant has devised, tested and embodied the present invention to overcome the shortcomings of the state of the art and to obtain this and other purposes and advantages.

### SUMMARY OF THE INVENTION

The present invention is set forth and characterized in the main claims, while the dependent claims describe other characteristics of the invention or variants to the main inventive idea.

In accordance with the above purposes, the device according to the invention comprises first containing means and second containing means, each having a specific function in a specific step of the method, which are arranged in a substantially integrated structure.

In the second containing means, in a first zone of analysis, a plurality of containers are arranged, inside each of which there is a biological sample to be analyzed. A eugonic broth, or eugonic cultural soil, mixed with the biological sample, is introduced into said containers, and is able to promote the bacterial growth for the purposes of the analysis.

The device also comprises, in the same integrated structure, first examination means used in a first step to examine the content of the containers containing the biological sample mixed with the eugonic broth. The first examination step, or screening, allows to verify the presence or absence of bacteria in the sample and, if affirmative, to identify at least the type of bacteria. This identification takes place at least according to the morphology of the bacteria, dividing them for example between cocci, in which morphologically the spherical form prevails, and bacilli, in which morphologically the stick shape prevails.

In the integrated structure there are also second examination means able to verify, in a second zone of analysis of the second containing means and in a second step performed when the bacteria has grown, the response of each positive biological sample, enriched by the presence of grown bacteria, to a series of antibiotics of a group of antibiotics chosen according to the type of bacterium identified.

The analysis thus performed is automated and substantially does not require the intervention of any operator while it is performed. The analysis provides rapid results based on the response, sensitive or resistant, of the bacterium to the series of antibiotics tested.

According to a variant, the first containing means comprise a cooling unit with the function of keeping the characteristics of the pure biological samples unchanged, preventing the relative bacterial charge from being modified.

According to another variant, the second containing means comprise a heating unit associated with the first and second zone of analysis. The heating unit, together with the function performed by the eugonic broth, promotes and accelerates the bacterial growth of the positive biological samples.

According to another variant, the positive biological samples are kept stirred by means of stirring means.

In another variant, in the same integrated structure, the device comprises automatic selection means able to pick up a desired quantity of a specific biological sample.

In a first step, the quantity of sample is picked up by a test tube and dispensed in a corresponding container located in the first zone of analysis; in a second step, a quantity of sample is picked up from a container of the first zone of analysis and dispensed, or divided, into one or more containers located in the second zone of analysis.

The selection means comprise at least a pick-up and dispensing device supplied with needle means and gripping means able to be activated on a test tube or container.

The device according to the invention also comprises a control unit able to control and command at least the selection means, and the first and second examination means. The control unit can be arranged irrespectively inside the integrated structure, or outside it.

According to the invention, at least the first and advantageously also the second examination means comprise means to emit electromagnetic radiations, for example coherent light, and means to detect said electromagnetic radiations. The emitter means and the detection means are arranged substantially on a circumference at the center of which, according to the examination step in progress, there is the container containing the biological sample to be classified, or the container containing the biological sample which has already been classified with regard to type of bacterium and which is to be subjected to the sensitivity test to antibiotics.

The first and second examination means provide curves showing the growth of the concentration of the bacterium according to time and, according to these curves, the control unit verifies the presence of bacteria, identifies the type and identifies the antibiotics for a possible antibiotic therapy. The growth curves also describe the morphology of the bacterium.

If the original sample does not contain any bacterial charge, the result of the first examination step is a straight line (that is, zero growth) and the output of the device is a “negative result” which states that the sample under examination is not contaminated by any bacterial colony.

Only the “positive sample”, in which the presence of a bacterial growth has been ascertained, are made to pass to the second step, i.e. the step of identification of the species of the bacteria by using the different series of antibiotics, as explained in detail in the following.

According to a variant, a verification or counter-examination step is provided, in order to evaluate that the examination has been performed correctly. To this end, inside the integrated structure, the device comprises third examination means able to analyze the spectral content of a gas produced by each positive biological sample.

According to another variant, a verification step is provided, after the first examination step, which provides to mix a reagent substance, for example potassium hydroxide, with one or more biological samples, and to analyze the reaction times of each of the biological samples with said reagent substance.

### BRIEF DESCRIPTION OF THE DRAWINGS

These and other characteristics of the present invention will become apparent from the following description of a preferential form of embodiment, given as a non-restrictive example with reference to the attached drawings wherein:

FIG. 1 is a schematic view of an integrated device according to the present invention for diagnostic analyses;

FIG. 2 is a schematic view of a detail of the device in FIG. 1;

FIG. 3 is a schematic view of another detail of the device in FIG. 1;

FIG. 4 is a schematic view of another detail of the device in FIG. 1;

FIG. 5 is a schematic view of a variant of FIG. 3;

FIG. 6 is a schematic view of a variant of FIG. 2;

FIG. 7 is a flow chart of a method according to the present invention for diagnostic analyses;

FIG. 8 shows a variant of the device in FIG. 1.

### DETAILED DESCRIPTION OF A PREFERENTIAL FORM OF EMBODIMENT

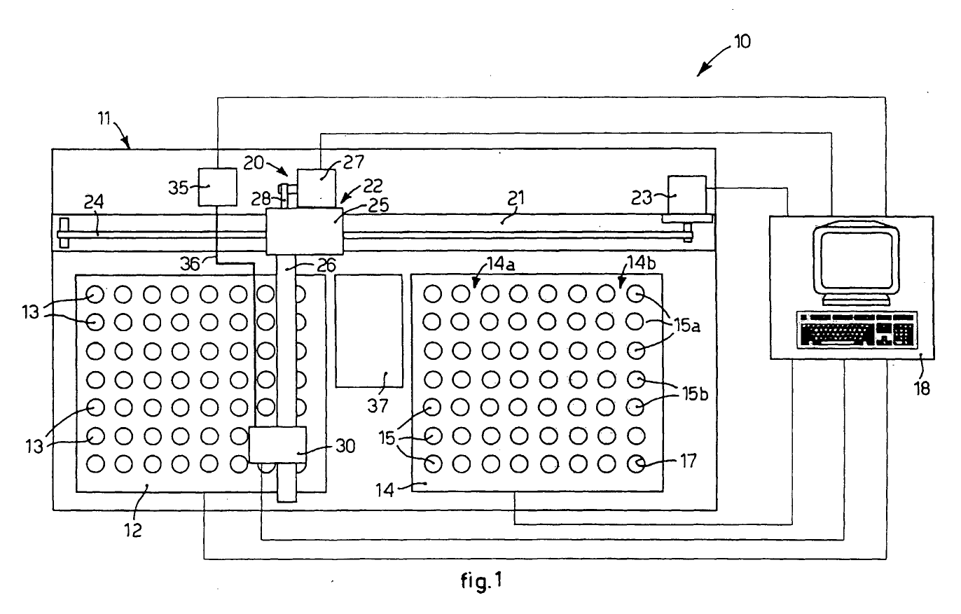

With reference to FIG. 1, an integrated device 10 for diagnostic analyses according to the invention comprises, in an integrated structure 11, a first container 12 containing a plurality of test tubes 13, inside each of which there is a pure biological sample, for example urine, cerebrospinal fluid, catarrh or diluted blood.

The first container 12 is associated with a cooling unit, not shown here, which takes or keeps the temperature of the pure biological samples within a range of between about 2° and 8° C., to prevent any variation in the characteristics of the biological samples and to keep the bacterial charge stable.

The device 10 also comprises a second container 14 containing, in a first zone of analysis 14a, a plurality of culture containers 15 arranged in relative seatings 17.

The second container 14 is associated with a heating unit, not shown here, to heat the biological samples to be analyzed to a temperature of between about 35° C. and 37° C., in order to promote the bacterial growth of any possible bacteria present.

The above term “any possible” means that the biological samples contained in the test tubes 13 have not been previously screened between positive and negative samples, but are pure samples directly coming from a step of drawing the sample from a patient.

A control unit 18, for example an electronic calculator, which can be either inside or outside the integrated structure 11, is associated with the integrated device 10.

The integrated device 10 also comprises a movement and selection unit 20, controlled by the control unit 18, consisting of a guide 21 on which a mobile support 22 moves in linear manner, moved by a first motor 23 by means of a first belt 24. The mobile support 22 comprises a head 25, with which an arm 26 is constrained, associated with a second motor 27 able to move, by means of a second belt 28, a selection head 30 free to slide on the arm 26.

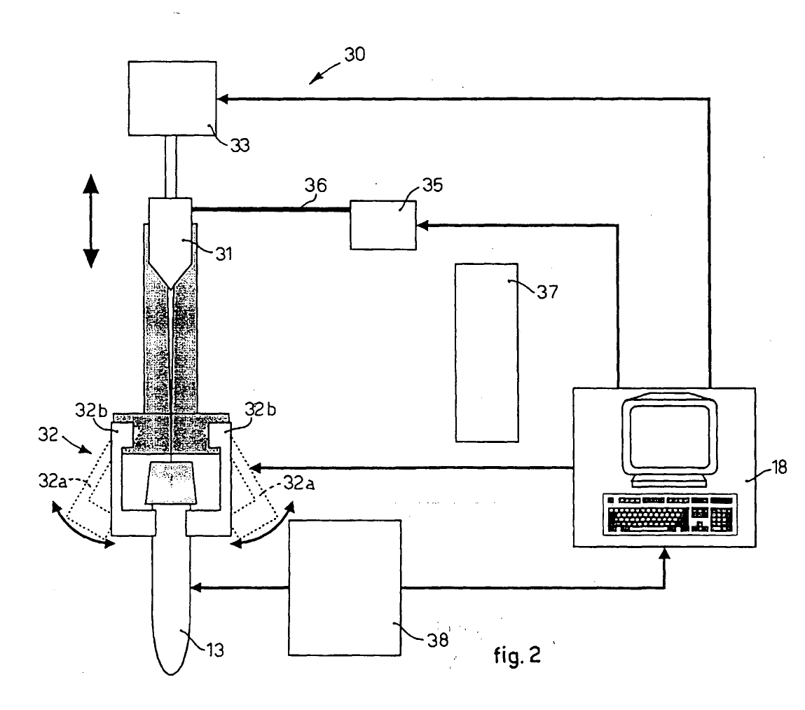

The selection head 30 (FIG. 2) comprises a pick-up and dispensing needle 31, a gripper 32 to grip the test tube 13 or container 15, and an actuator 33 able to selectively move the needle 31 and the gripper 32. The gripper 32, to be more exact, has an open position 32a and a closed position 32b to constrain the test tube 13 or container 15, for example in order to displace the latter from the first container 12 to the second container 14.

The selection head 30 is connected to a pumping mechanism 35 by means of a pipe 36, advantageously of the flexible type, for example made of rubber. The control unit 18 drives the pumping mechanism 35 to pick up and dispense, by means of the needle 31, a desired quantity of biological sample.

The integrated device 10 also comprises a washing zone 37, consisting for example of a tub, for the internal and external sterilization of the needle 31 which is advantageously performed after every operation to pick up and dispense the biological sample, so as to prevent any contamination of the bacterial charge between the different biological samples picked up and dispensed.

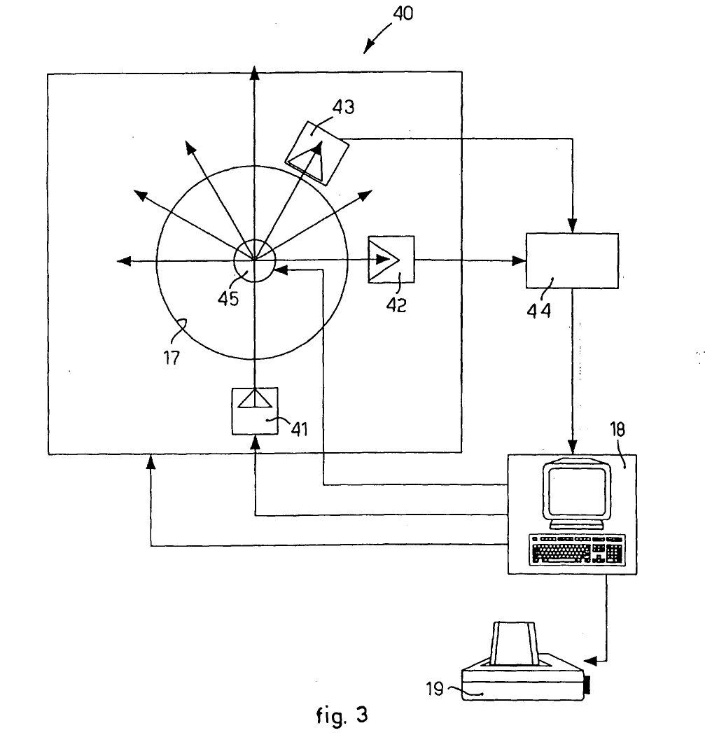

The second container 14 comprises, advantageously for every seating 17 of the first zone of analysis 14a, a first examination device 40 (FIG. 3), of a known type, having a laser emitter 41, with which a first sensor 42 and a second sensor 43 are associated, arranged respectively at about 90° and 150° with respect to the laser emitter 41 and able to detect the light that, emitted by the laser emitter 41, passes through the container 15.

The data collected by the first 42 and second sensor 43 are sent to the control unit 18 by means of a conditioning device 44, which amplifies, filters and processes the data collected.

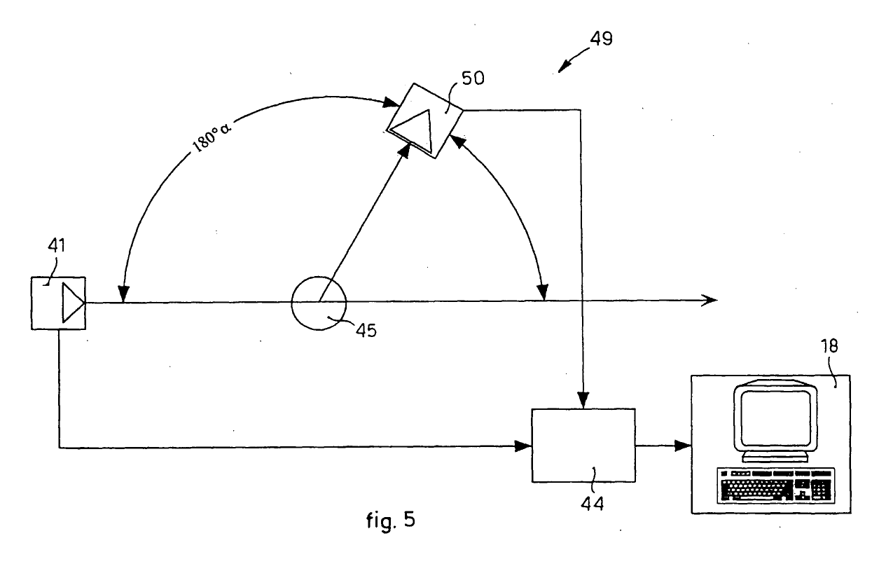

The second container 14 also contains, advantageously for every seating 17 of a second zone of analysis 14b, a second examination device 49 (FIG. 5), of a known type and similar to the first examination device 40. The second examination device 49 comprises a laser emitter 41 with which a single sensor 50 is associated, movable on a circumference arc which subtends an angle of about 180°, and moved by a motor, driven by the control unit 18 and not shown in the drawings.

In this case too the data collected by the sensor 50 are sent to the control unit 18 by means of the conditioning device 44.

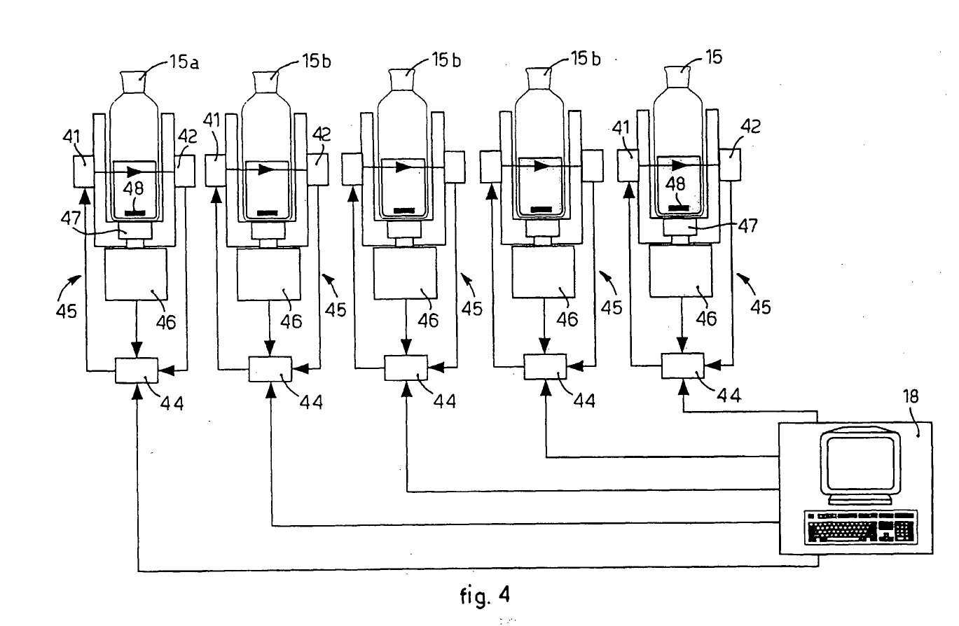

Every first and second examination device 40 and 49 also comprises a stirrer unit 45 (FIG. 4), equipped with a stirrer motor 46 controlled by the control unit 18, in order to make a first magnet 47 rotate, mechanically connected to the stirrer motor 46, and able in turn to make a second magnet 48 rotate, inserted inside the corresponding container 15 so as to mix the content thereof.

The integrated device 10 as described heretofore operates according to a method, indicated generally by the reference number 60 in FIG. 7, which provides the following steps.

In a first pick-up and dispensing step 61, the control unit 18 drives the movement and selection unit 20 in order to pick up a desired quantity of a specific pure biological sample from the respective test tube 13 and to dispense said quantity into a container 15 arranged in the first zone of analysis 14a, sterilized and inside which there is a eugonic broth.

The eugonic broth can already be present inside the container 15 before the biological sample is dispensed, or it can be inserted afterwards. The growth of the bacteria possibly present occurs in the container 15.

When the first pick-up and dispensing step 61 is terminated, there follows an identification step 62 during which the control unit 18 activates the first examination devices 40 so that the sensors 42, 43 of each device 40 periodically detect the laser emissions emitted periodically by the laser emitter 41.

The biological samples, in the presence of duplicating bacteria, emit signals of diffused light which the control unit 18 processes in order to supply, starting from about 45 minutes from the start of incubation, specific curves which express the development of the bacterial growth over time.

From the signals supplied by the two sensors 42 and 43, two curves are obtained of the growth of the possible bacterium, having respective slopes and a reciprocal divergence which make possible to verify the presence of the bacterium and to identify its type.

Subsequently, the control unit 18 identifies the bacteria belonging to the coccus type, which have a reciprocal divergence of the growth curves which allows them to be distinguished from the bacillus type.

The signal obtained from the second sensor 43 defines a first curve relating to the development of the bacterial charge over time, correlated to the type of bacteria classified as cocci or bacilli. Moreover, the relation between the signals obtained from the second 43 and the first sensor 42 defines a second curve leading to the type of bacterium and particularly to its morphology.

Therefore, with the first examination device 40 the control unit 18 verifies the presence of bacteria in a corresponding container 15 and, if affirmative, identifies the type by analyzing the relation between the signals obtained by the second sensor 43 and the first 42.

The sensitivity thresholds of the count of the bacterial growth start from about 50 cfu (colony forming unit)/ml, that is, the number of units forming a colony per millimeter of biological sample, up to about 100 million cfu/ml. The integrated device 10 is therefore able to perform a diagnostic analysis with a sensitivity range varying according to the type of sample, either sterile or from midstream.

The control unit 18 is connected to an output device 19 (FIG. 3), in this case a printer, or an external memorization device, not shown here, such as for example a hard disk, a floppy disk, respectively to print and memorize at least the data concerning the curves supplied by the control unit 18. The latter also memorizes the curves according to type of growth with respect to the bacteria identified in order to supply a databank for confrontation and/or comparison for every examination performed.

Moreover, the control unit 18, by means of the first examination device 40, verifies the suitability of the biological samples for analysis, for example by evaluating the turbidity thereof, signaling the possible non-suitability by means of the output device, and/or by means of an acoustic signaler.

When the identification step 62 is terminated, there follows a second pick-up and dispensing step 64, during which the control unit 18 drives the movement and selection unit 20 in order to pick up the positive biological samples, enriched by the presence of grown bacteria, recognized as such during the previous identification step 62, in order to dispense them into a group of first 15a and second 15b containers, located in the second zone of analysis 14b.

During this step, according to a variant, it is possible to use a measuring instrument (not shown in the drawings) to standardize the concentration of the bacterial suspension taken, which will then be used to carry out the sensitivity test to antibiotics and the identification of the bacteria.

To this purpose, a preferential embodiment of the invention provides to apply to the movement and selection unit 20 an instrument to measure the turbidity of the bacterial suspension, in order to quantify the concentration thereof according to a standardized scale, for example the one known as the McFarland scale.

The concentration of bacteria according to this scale is constructed using a photometer that uses a radiation, normally in the range of 500-700 nanometers, which passes through the bacterial suspension and is detected on the opposite side. Each interval of the McFarland scale corresponds to an interval of absorbance. In this way, with a turbidity scale correlated to the McFarland scale, it is possible, in the pick-up and dispensing step, to standardize the concentration of the bacterial suspension taken, thus obtaining more reliable results in the subsequent steps of carrying out the sensitivity test to antibiotics and identifying the bacteria. To give an example, for this purpose it is possible to use the examination device 40 as a device to measure the turbidity correlated to the McFarland scale.

Each positive biological sample can be picked up from the biological sample that has grown in the eugonic broth contained in the respective container 15 of the first zone of analysis 14a, or directly from the pure biological sample contained in the corresponding test tube 13, in this case without the eugonic broth.

To be more exact, in each of the first containers 15a only the corresponding positive biological sample dispensed is present, which is also called the reference sample, while inside each of the second containers 15b there is also an antibiotic. The control unit 18 identifies each of these antibiotics according to the type of bacteria determined, identified during the identification step 62.

Each of the antibiotics is present in liquid form and is ready for dispensing, or is prepared there and then, so as to be optimized in the final concentration ready for action.

After the second pick-up and dispensing step 64 there follows the step of the sensitivity test to antibiotics 65, during which the control unit 18, by means of the first examination devices 40, analyses the growth curves of the bacteria both of the reference sample and also of the biological samples contained in the containers 15b and treated with different antibiotics.

To be more exact, the control unit 18 compares the growth curves of the reference sample with the growth curves, or inhibition curves, of the biological samples treated with different antibiotics, in order to verify the effectiveness of the antibiotic.

The analysis of said growth curves or inhibition curves, for example like the corresponding inhibition haloes of the Kirby-Bauer method, determines the effectiveness of the antibiotic, in vitro, by means of the functions, respectively, resistant (R), sensitive (S) or intermediate (I), which respectively indicate how much the bacterium resists the antibiotic and how much it is sensitive thereto.

The curves can be represented graphically, and printed by the output device 19, and express the percentage of effectiveness in the antibiotic treatment required for every clinical type or request for verification.

The percentage of effectiveness of the antibiotic in relation to the specific biological sample is expressed in a percentage from 0% (S=sensitive) to 100% (R=resistant) with respect to the reference biological sample, to which, as explained, no antibiotic has been added.

The control unit 18 also examines the number of units forming colonies per millimeter of biological sample, cfu/ml, and for every specific biological sample, and based on pre-defined data, associates this cfu value with an appropriate quantity of antibiotic to dispense, in a manner correlated to the bacterial charge.

In this way, the control and verification of the functionality of the antibiotics are particularly correct from the therapeutic point of view, given that the function of an antibiotic is correlated to the quantity of bacteria present in the biological sample itself.

In one embodiment, in order to make the best choice of antibiotics with respect to the type of bacteria, a verification step 63 is provided, performed after the identification step 62 and before the second pick-up and dispensing step 64.

The verification step 63 is performed on every biological sample in order to verify, in a first substep, the correct identification made by means of analyzing the relation of the slope of the curves revealed. The verification step allows, as a hypothesis, that bacteria of the coccus type correspond to the bacteria classified as Gram+, and bacteria of the bacillus type correspond to bacteria classified as Gram−. This hypothesis is valid at least as far as regards the analysis of infections of the lower urinary tract.

This first substep provides to identify the type of bacteria and particularly the bacterial class GRAM− and GRAM+, for example according to the known Halebian method. According to this method, the GRAM+ and GRAM− bacteria react in the presence of potassium hydroxide KOH at 3%, forming a lysis of the bacterial membrane in a selective manner. To be more exact, the GRAM− bacteria lysed after the addition of KOH make the culture broth viscous, unlike GRAM+ bacteria, which reach this state after a longer time.

The control unit 18, for example by means of the first examination devices 40, examines the viscosity of the biological samples in relation to time and, based on the differential times, recognizes the types of bacteria to confirm the previous typological analysis of the growth curves, as made during the identification step 62.

During a second substep, the control unit 18, only on the positive samples, performs an analysis of the samples no longer by means of the first examination device 40, but by means of the second examination device 49, obtaining a reading over the whole angle of 180°. This amplitude of reading allows to detect all the variables of the diffusion of the laser, allowing to construct growth curves with characteristics easily identifiable for every type of bacteria.

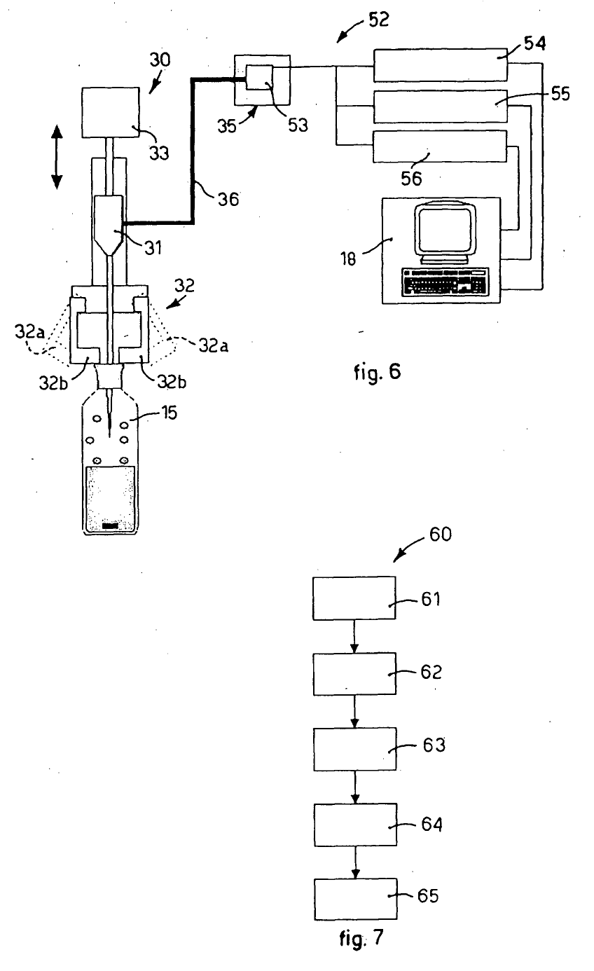

In another solution, the integrated device 10 also comprises, in the integrated structure 11, a third examination device 52 (FIG. 6) comprising a reading cell 53, in this case inside the pumping mechanism 35, and a mass spectrometer 54, a spectrophotometer 55, for example infra-red, and a gas chromatograph 53.

When the bacterial growth has taken place and been detected, the control unit 18, during a third substep, drives the movement and selection unit 20, so that the needle 31 perforates a stopper that hermetically closes a respective container 15, 15a, 15b.

By means of the pumping mechanism 35 a desired quantity of gas present in the volume between the biological sample and the stopper of the respective container 15, 15a, 15b is picked up. This quantity of gas is transferred to the reading cell 53, so that verification can take place by means of the mass spectrometer 54, the spectrophotometer 55, or the gas chromatograph 53.

The invention allows to perform the cultural analysis of the bacteria present in biological samples of any nature or origin, including swab samples, for example in hospital environments, of particular interest for safeguarding the environmental hygiene.

The results can be obtained within about 24 hours from when the biological sample is inserted into the integrated device 10, and automatically. Moreover, the clinical reports can be printed automatically and memorized in the form of a databank.

It is clear that modifications and/or additions of parts and/or steps may be made to the integrated device 10 and method 60 as described heretofore, without departing from the field and scope of the present invention.

For example, it may be provided that the whole test tube 13 is transported into the second container 14, and the subsequent steps of analysis are performed on said test tube 13. Moreover, the test tubes 13 containing the biological samples can be arranged directly in the second container 14.

It is also provided that a motor can be associated with the first container 12, in order to impart a vibratory movement to mix the content of the test tubes 13.

The first container 12 can have a cylindrical or similar shape, and have lateral seatings on the surface for the corresponding test tubes 13.

It may also be provided that the integrated device 10, by means of the control unit 18, can verify the residual antibiotic power (RAP) in a particular biological sample, in order to ascertain whether the patient to whom the determinate biological sample refers is taking antibiotics or not.

According to another variant, the second examination device 49 can be arranged in correspondence with the first zone of analysis 14a, to verify the presence and identify the type of bacteria.

It may also be provided to arrange, in every seating 17, a reading device 38 (FIG. 2), for example a bar code reader, controlled by the control unit 18. The reading device 38 can read a bar code printed on a label on each of the containers 15, 15a, 15b, so as to univocally identify the container 15, 15a, 15b, the biological sample contained therein, and consequently the patient from whom the biological sample has been taken.

It is also provided that the control unit 18 can memorize the displacements, samplings and dispensing performed by means of the movement and selection unit 20. In this way the content of any container 15, 15a, 15b can always be correlated to the respective patient.

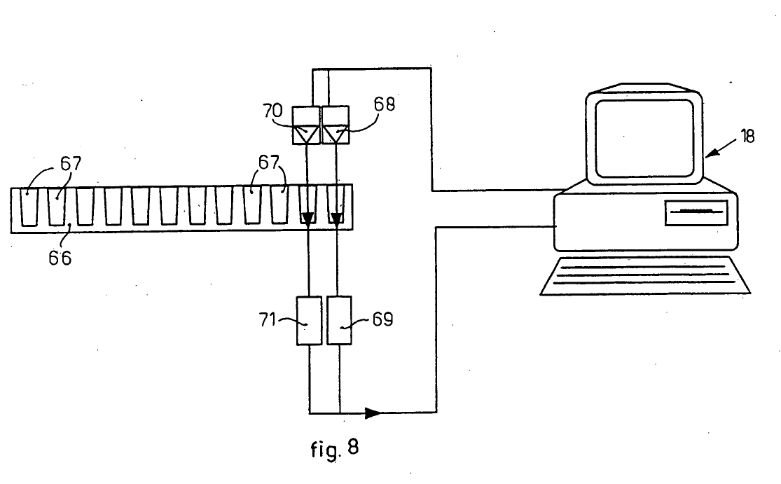

According to the variant shown in FIG. 8, instead of the group of containers 15 shown in FIG. 4, a plate 66 is used, of a standardized type, comprising a plurality of recesses 67, which function as containers for the bacterial growth and for the biochemical reactions described above.

The plates 66 of a standardized type comprise 96 or 384 recesses 67 and their use allows to drastically reduce the overall bulk of the device with respect to a similar device that uses the cylindrical containers 15.

A standard plate with 96 recesses occupies a surface of cm 8.5×12.5 with cylindrical recesses 67 sized mm 7×9. A similar plate with 384 recesses occupies the same surface but each recess 67 can contain at most 80 microliters.

The use of such small-size plates 66 not only gives the advantage of producing a smaller quantity of potentially infected material, but also allows to effect biochemical reactions to identify the bacterial species.

To this purpose, one of the recesses 67 will be filled with a reference culture and a number of other recesses 67 will be filled with the same bacterial suspension to which will be added a suitable concentration of a different antibiotic in order to select the most suitable one.

It will be possible to evaluate the kinetics of growth or inhibition in each recess 67 over some hours, using a system to detect the turbidity consisting of a light source 68 facing which, on the opposite side of the plate 66, there is a turbidimeter 69.

The large number of recesses available also allows to fill others with the same bacterial suspension into which, in every recess 67, a different chemical reagent will be introduced. These different chemical reagents will cause, in the series of recesses 67, a different combination of colors connected to a particular bacterial species. The combination of colors can be detected by means of a sensor comprising a light source 70 disposed facing, on the opposite side of the plate 66, a CCD camera 71, or other suitable sensor. The data detected can then be transmitted to the control unit 18 which, by means of suitable algorithms, discriminates the bacterial species according to the resulting combination of colors.

1. An integrated device for diagnostic analyses used to verify the presence of bacteria in at least a biological sample mixed with a eugonic culture broth, in order to identify at least the type of bacteria, and to test a series of antibiotics, selected from a group of antibiotics characteristic at least for said type of bacteria identified, identifying those effective to determine the antibiotic therapy, the device comprising inside an integrated structure, first optical examination means, having a laser emitter, able to verify the presence of bacteria, by observing the related growth curve, to define corresponding positive biological samples, and to identify at least the type, by observing the development of the bacterial growth over time of bacteria present in said positive biological samples, in order to define said group of antibiotics, and second examination means able to verify the sensitive or resistant response of each positive biological sample to a series of antibiotics of said group of antibiotics defined by said first examination means.

2. An integrated device as in claim 1, wherein a desired quantity of each positive biological sample is directly mixed with at least an antibiotic of said series of antibiotics, in order to verify the sensitivity or resistance of the bacterium to said antibiotic with respect to a desired quantity of said positive or reference sample, which is not mixed with any antibiotic.

3. An integrated device as in claim 1, comprising first and second containing means arranged inside said integrated structure, wherein said second containing means define a first and a second zone of analysis, said first examination means being associated at least with said first zone of analysis and said second examination means being associated with said second zone of analysis.

4. An integrated device as in claim 3, wherein said first containing means are able to contain a plurality of test tubes inside each of which a pure biological sample is present, cooling means being associated with said containing means in order to ensure the correct preservation of said pure biological samples.

5. An integrated device as in claim 3, wherein said second containing means comprise a heating unit able to heat said biological samples in order to promote the bacterial growth.

6. An integrated device as in claim 3, comprising selection means able to pick up a desired quantity of a pure biological sample contained in a test tube in order to dispense said desired quantity into a specific container containing eugonic broth and arranged in said first zone of analysis, associated with said first examination means.

7. An integrated device as in claim 6, wherein said selection means are able to pick up a desired quantity of a determinate positive biological sample contained in a container, arranged in said first zone of analysis, so as to divide said desired quantity into a plurality of first and second containers arranged in said second zone of analysis.

8. An integrated device as in claim 7, wherein said selection means cooperate with means to standardize the bacterial concentration present in the suspension taken.

9. An integrated device as in claim 8, wherein said standardization means comprise a photometer able to detect the turbidity of the suspension and to classify the concentration thereof according to the McFarland scale.

10. An integrated device as in claim 7, wherein an antibiotic of said group of antibiotics characteristic for at least said type of bacteria identified by said first examination means is introduced inside each of said second containers, wherein said determinate positive or reference biological sample is present substantially exclusively inside each of said first containers.

11. An integrated device as in claim 10, wherein said second examination means are able to compare the development of the bacterial charge of the biological samples contained in every container of said second plurality of containers with the development of the bacterial charge of the corresponding determinate positive biological sample contained in said first containers.

12. An integrated device as in claim 1, wherein each of said first and second examination means comprises said emitter means to emit electromagnetic radiations, and detection means to detect said electromagnetic radiations which pass through said container.

13. An integrated device as in claim 12, wherein the detection means of said first examination means include at least two fixed sensor elements, wherein the detection means of said second examination means include at least a movable sensor element.

14. An integrated device as in claim 12, wherein the detection means of the first and second examination means include at least a movable sensor element.

15. An integrated device as in claim 13, wherein said fixed sensor elements are arranged respectively at about 90° and 150° with respect to said emitter means and along a circumference at center of which said container is arranged.

16. An integrated device as in claim 1, further comprising, in said integrated structure, third examination means able to examine the positive biological samples in order to evaluate the correctness of the examination performed by the first examination means or the second examination means.

17. An integrated device as in claim 16, wherein said third examination means comprise devices able to analyze the spectral content of a gas produced by every positive biological sample.

18. An integrated device as in claim 6, wherein said selection means comprise mechanisms to move at least a pick-up and dispensing needle.

19. An integrated device as in claim 18, further comprising, in said integrated structure, a washing and sterilizing device, to wash and sterilize said pick-up and dispensing needle.

20. An integrated device as in claim 6, comprising reading means able to identify each of said containers, in order to correlate each of said containers to the biological sample contained therein and to the patient from whom said biological sample was taken.

21. An integrated device as in claim 6, further comprising a control unit able to at least memorize the displacements, the samples and the dispensing performed by means of said selection means.

22. An integrated device as in claim 1, comprising, in said first analysis zone, a plate defining a plurality of recesses each of which is able to be filled with the bacterial suspension and a relative antibiotic in order to select the most suitable antibiotic for the specific bacterium.

23. An integrated device as in claim 22, comprising means to detect the turbidity able to detect the kinetics of growth or inhibition in each of said recesses containing the bacterial suspension and a relative antibiotic.

24. An integrated device as in claim 22, wherein a chemical reagent is added to the bacterial suspension in at least some of said recesses, the device comprising means to detect the combination of colors produced by said chemical reagents in order to discriminate the bacterial species according to the resulting combination of colors.

25. A method for diagnostic analyses used to verify the presence of bacteria in at least a biological sample mixed with a eugonic culture broth, in order to identify at least the type of bacteria, and to test a series of antibiotics, selected from a group of antibiotics characteristic at least for said type of bacteria identified, identifying those effective to determine the antibiotic therapy, the method comprising the following steps:
a first examination step during which the content of a plurality of biological samples is examined by means of an optical examination device comprising at least a laser emitter, to verify the presence of bacteria to define a plurality of positive biological samples, and to identify at least the type of bacteria to define said group of antibiotics;
a second examination step, during which, if the result of the first examination step is affirmative, the sensitive or resistant response is verified of each positive biological sample to a series of antibiotics of said group of antibiotics defined in said first examination step.

26. A method as in claim 25, wherein during said second examination step each positive biological sample is mixed with at least an antibiotic of said series of antibiotics, in order to verify the sensitivity or resistance of the bacterium to said antibiotic with respect to a positive reference sample, to which no antibiotic has been added.

27. A method as in claim 25, further comprising a first selection step performed before said first examination step, during which a desired quantity of a pure biological sample is taken, contained in a respective test tube arranged in first containing means, and is dispensed in a container, arranged in second containing means, and containing said eugonic broth so as to promote the bacterial growth.

28. A method as in claim 25, further comprising a second selection step, performed after said first examination step and before said second examination step, during which a desired quantity of a specific positive biological sample is taken, enriched by the presence of grown bacteria, in order to divide it said desired quantity into a plurality of second containers inside each of which an antibiotic of said group of antibiotics is provided characteristic at least for said types of bacteria.

29. A method as in claim 25, further comprising a verification step after said first examination step, to verify the correct identification of the type of bacteria as resulting from said first examination step, which provides to mix a reagent substance with one or more of said biological samples, and to analyze the reaction times of each of said biological samples with said reagent substance.
